(19) United States
(12) Patent Application Publication
(10) Pub. No.: US 2024/0221157 A1
(43) Pub. Date: Jul. 4, 2024

(54) METHOD FOR PROVIDING SUPPLY DATA RELATING TO THE SUPPLY OF A PARENCHYMA

(71) Applicant: Siemens Healthcare GmbH, Erlangen (DE)

(72) Inventors: Bernd HOFMANN, Erlangen (DE); Hendrik DITT, Neustadt an der Aisch (DE); Matthias LICHY, Nuernberg (DE); Chris SCHWEMMER, Forchheim (DE)

(73) Assignee: Siemens Healthcare GmbH, Erlangen (DE)

(21) Appl. No.: 18/522,578

(22) Filed: Nov. 29, 2023

(30) Foreign Application Priority Data
Dec. 29, 2022 (DE) 10 2022 214 444.9

(51) Int. Cl. G06T 7/00; A61B 5/02; A61B 5/021; A61B 5/107; G06T 7/62; G16H 30/40

(52) U.S. Cl. CPC G06T 7/0012; A61B 5/02028; A61B 5/021; A61B 5/1076; G06T 7/62; G16H 30/40; G06T 2207/30104

(57) ABSTRACT

A method for providing supply data relating to supply to a parenchyma, comprises: receiving first imaging data, wherein the first imaging data relates to the parenchyma and/or a vascular structure that serves to supply the parenchyma; receiving reference data for the supply to the parenchyma; calculating the supply data based on the first imaging data and the reference data, wherein the supply data relates to the supply to the parenchyma; and providing the supply data.

### CROSS-REFERENCE TO RELATED APPLICATION (S)

The present application claims priority under 35 U.S.C. § 119 to German Patent Application No. 10 2022 214 444.9, filed Dec. 29, 2022, the entire contents of which is incorporated herein by reference.

### FIELD

One or more example embodiments of the present invention relate to a method for providing supply data relating to the supply of a parenchyma. One or more example embodiments of the present invention further relate to a data processing system and a medical imaging system.

### BACKGROUND

While the vascular supply of most organs is ensured by a continuous branching of the arterial vessels, the supply of some organs is ensured by ring-shaped vascular structures fed by a plurality of vessels or at least by double vascular structures. The purpose of these anatomically special structures is to ensure the supply of particularly important organs, even if one of the supplying vessels becomes occluded. The prime example of these peculiarities can be seen in the brain. On the one hand, the Circulus Arteriosis Wilisii/Circle of Willis (CAW) allows the blood flow to be distributed from the four arteries supplying the brain (arteriae carotis internae and vertebrales) via the two cerebral hemispheres and parts of the cerebellum to the cerebral arteries.

Another safety mechanism, the so-called collaterals, ensure that, after a period of adaptation, blood flow can occur between the supply zones of the large cerebral arteries via small vascular connections on the surface of the brain and thus the occlusion of one of these large vessels (leaving from the circle) can at least theoretically be compensated for. However, a sudden vascular occlusion often cannot be compensated for due to the need for adaptation of the collaterals.

Due to the increasing frequency of vascular diseases and the resulting increased risk of strokes, it is not only important to diagnose vascular stenoses, but also to estimate their effects on future changes in blood flow or blood pressure in order to then derive indications for therapy. However, current imaging techniques, such as CT angiography (CTA) and perfusion CT (PCT), can only capture an instantaneous image of the condition.

As part of the currently common diagnostic measures, CTA usually takes place as standard. When examining the anatomy of the vessels, the diameter and contrast medium (CM) patency are taken into account. If there are larger differences in flow, differences in density can also be an indication of lower perfusion. Measuring density differences is prone to errors when evaluated manually and is very dependent on external parameters.

A variant of CTA is 4D CTA, which is used in particular when an exact time estimate of the optimal filling of the vessels cannot be made. Although CTP, which has an even higher temporal resolution, can in principle be used to assess long vascular sections, it does involve some technical effort. If there is a pre-existing, especially incomplete, stenosis in one of the large cerebral arteries, e.g. the middle cerebral artery, and a normal finding in the perfusion CT, in many cases it is not possible to estimate how the supply to the downstream brain tissue changes under other physiological prerequisites.

A typical application is the assessment of vascular pathology of the cerebral vessels or the feeding vessels. It should be noted that changes in the vessel cannot occur in the immediate vicinity of the territorial final flow zone. The case of a single or multiple (partial) occlusion, the effects of a possible drop in blood pressure or progression of the vascular occlusion in relation to the territorial tissue supply must be considered.

In contrast to coronary heart disease or renal artery stenosis, there is no direct connection between the degree of stenosis and territorial deficiency. This has to do with the above-mentioned anatomical peculiarity of the cerebral supply, which has bypass circuits. It is therefore possible that the downstream area, e.g. the middle cerebral artery, is supplied by already sufficiently opened collaterals, e.g. from the supply zone of the anterior cerebral artery. However, it may also be the case that the stenosis is not yet so severe and the area is adequately supplied via the stenotic vessel without the collaterals from the neighboring region already being functionally opened.

If there is an acute drop in supply in the zone distal to the stenosis, e.g. due to a rapid drop in blood pressure, in the first case the supply from the neighboring region can probably be at least to such an extent that ischemia with cell death does not occur. In the second case, adequate supply would not be guaranteed because the collaterals first have to expand and thus ischemia occurs in the supply zone behind the stenosis.

In order to prevent an ischemic event, one would consider placing a vascular stent or a bypass over the stenosis in the second case, while this is not necessary in the first case. For the therapeutic option of vascular bypass, it is also interesting to estimate the extent to which sufficient perfusion can be achieved. It should be noted that the starting points may sometimes not be in the immediate vicinity of the vascular change. The exact indication is necessary both for cost reasons and for reasons of the not insignificant risk of complications when placing stents in the narrow vessels of the cerebral circulation. Currently, prophylactic therapy is usually used.

Determining the pressure gradient between an area in front of the stenosis and a sector behind the stenosis can help to estimate the success of treatment or the need for treatment. Longer vascular anomalies of the cerebral vessels, such as vasospasm or pathological changes such as moyamoya disease, should be considered as further special cases. Since, unlike the heart, for example, there is no sequential blood flow in the brain via branching arteries, the classic Fractional Flow Reserve (FFR) approach to pressure measurement has disadvantages that result from the fact that the previous approach does not take the supply from a plurality of vessels into account.

In addition to the peculiarity described above, there is another one. It's not just the blood flow in the immediate environment around the stenosis that can have an impact on reduced blood flow to the distal areas of the brain. The flow dynamics in a vascular circle are not always comprehensively represented using current methods. A reduction in blood flow in feeding arteries (e.g. due to a drop in blood pressure) can have an impact on the flows in the CAW and thus on the pre-stenotic pressure of the affected cerebral artery, among other things, but not only, if an incomplete CAW is present. A sole consideration of the conditions surrounding the stenosis is therefore not sufficient to make a statement about the occurrence of ischemia in the brain tissue.

The same applies if there is a stenosis in one of the arteries supplying the brain, e.g. the carotid artery. In both cases, it is not enough to determine the input before the stenosis in the cerebral artery, but rather the pressure and flow conditions on the relevant or on all vessels supplying the brain must be included in the calculation, as well as on the downstream cerebral arteries, which directly supply the tissue. This calculation is also significantly more complex than with purely linear vessel relationships, as has previously been the basis for the FFR. Likewise, vascular calcification can influence flow dynamics and autoregulation.

Spin labeling and phase contrast techniques in MRI provide options for assessing vascular territories. However, these methods do not easily allow precise quantification of the flow parameters. Ultrasound procedures can also be considered to assess flow differences and restrict stenosis. Here, too, these are user-dependent and more complex techniques that generally cannot cover the entire vascular zone. CT angiography enables an anatomical representation of the vessels, but it is not easy to provide quantifiable statements based on the morphology on the question described above.

Using invasive FFR, statements can be made about the capacity of the vessel in certain flow zones. In simple vascular systems such as the coronaries, additional computer simulations for specific FFR can be created based on angiography data. The previous FFR methods are not suitable for complex vascular zones (multi-wave/compensatory regulation in the terminal zone), such as those required in particular for the brain.

### SUMMARY

An object of one or more embodiments of the present invention is to enable an alternative to the conventional provision of supply data relating to the supply of a parenchyma. Each subject of an independent claim achieves at least this object. Further advantageous aspects of embodiments of the present invention are taken into account in the dependent claims.

Regardless of the grammatical gender of a particular term, it includes people with male, female or other gender identities.

An embodiment of the present invention relates to a method for providing supply data relating to the supply of a parenchyma, the method comprising:
receiving first imaging data, wherein the first imaging data relates to a vascular structure that serves to supply the parenchyma, and/or the parenchyma,
receiving reference data for parenchyma supply,
calculating the supply data, wherein the supply data relate to the supply of the parenchyma, based on the first imaging data and the reference data,
providing the supply data.

An embodiment of the present invention provides a data processing system for providing supply data relating to the supply of a parenchyma, having a data interface and a processor, wherein the data processing system is set up to carry out a method according to one or more embodiments of the present invention.

An embodiment of the present invention provides a medical imaging system comprising: the aforementioned data processing system; and a medical imaging device configured to record the first imaging data.

An embodiment of the present invention provides a non-transitory computer program product including instructions which, when executed by a computer, cause the computer to carry out a method according to an embodiment of the present invention.

An embodiment of the present invention provides a non-transitory computer-readable storage medium storing instructions which, when executed by a computer, cause the computer to carry out a method according to an embodiment of the present invention.

### BRIEF DESCRIPTION OF THE DRAWINGS

The present invention is explained below using exemplary embodiments with reference to the attached figures. The representation in the figures is schematic, highly simplified and not necessarily true to scale.

FIG. 1 shows a flowchart of a method for providing supply data relating to the supply of a parenchyma.

FIG. 2 shows a medical imaging system.

### DETAILED DESCRIPTION

Using invasive FFR, statements can be made about the capacity of the vessel in certain flow zones. In simple vascular systems such as the coronaries, additional computer simulations for specific FFR can be created based on angiography data. The previous FFR methods are not suitable for complex vascular zones (multi-wave/compensatory regulation in the terminal zone), such as those required in particular for the brain.

An object of embodiments of the present invention is to enable an alternative to the conventional provision of supply data relating to the supply of a parenchyma. Each subject of an independent claim achieves this object. Further advantageous aspects of embodiments of the present invention are taken into account in the dependent claims. Regardless of the grammatical gender of a particular term, it includes people with male, female or other gender identities.

An embodiment of the present invention relates to a method for providing supply data relating to the supply of a parenchyma, the method comprising:
receiving first imaging data, wherein the first imaging data relates to a vascular structure that serves to supply the parenchyma, and/or the parenchyma,
receiving reference data for parenchyma supply,
calculating the supply data, wherein the supply data relate to the supply of the parenchyma, based on the first imaging data and the reference data,
providing the supply data.

An embodiment of the present invention provides a data processing system for providing supply data relating to the supply of a parenchyma, having a data interface and a processor, wherein the data processing system is set up to carry out a method according to one or more embodiments of the present invention.

One embodiment provides that the vascular structure is ring-shaped. The vascular structure can, for example, comprise at least one main vessel and/or at least one collateral. The parenchyma can be, for example, a brain parenchyma. The vascular structure can, for example, supply the brain, in particular a Circle of Willis. The supply data can, for example, relate to a possible stenosis in the vascular structure and/or its effect on the supply of the parenchyma. In particular, the supply data can indicate whether there is collateralization of the vascular structure in such a way that the supply of the parenchyma is guaranteed even if the main vessel is occluded.

One embodiment provides that the reference data includes second imaging data, wherein the first imaging data relates to the vascular structure and/or the parenchyma at a first point in time, wherein the second imaging data relates to the vascular structure and/or the parenchyma at a second point in time. The reference data can in particular comprise comparison data from a database, for example in the form of a priori knowledge. The second point in time can, for example, be before or after the first point in time.

One embodiment provides that the flow dynamics of the vascular structure at the first point in time differs from the flow dynamics of the vascular structure at the second point in time, in particular substantially different, and/or that the perfusion dynamics of the parenchyma at the first point in time differ from the perfusion dynamics of the parenchyma at that second point in time, in particular differ substantially.

One embodiment provides that the vascular structure at the first point in time differs from the vascular structure at the second point in time with respect to a blood pressure and/or a vessel diameter, in particular differs substantially. This difference can be caused, for example, by a medication.

One embodiment provides that first blood pressure data, which relates to the blood pressure at the first point in time, and second blood pressure data, which relates to the blood pressure at the second point in time, are received, wherein the first blood pressure data and the second blood pressure data are compared with one another, in particular to determine a difference in the vascular structure at the first point in time from the vascular structure at the second point in time in relation to the blood pressure. The blood pressure data can, for example, be based on a blood pressure measurement.

One embodiment provides that a vessel diameter of the vascular structure is calculated at the first point in time based on the first imaging data, wherein a vessel diameter of the vascular structure is calculated at the second point in time based on the second imaging data, wherein the vessel diameter of the vascular structure at the first point in time is compared with the vessel diameter of the vascular structure at the second point in time, in particular to determine a difference in the vascular structure at the first point in time from the vascular structure at the second point in time in relation to the vessel diameter.

One embodiment provides that the supply data relates to the supply of the parenchyma at a third point in time, wherein the third point in time is after the first point in time and after the second point in time.

In particular, it can be provided that the first imaging data includes first vascular data and/or first parenchyma data, wherein the first vascular data relates to the vascular structure at the first point in time, wherein the first parenchyma data relates to the parenchyma at the first point in time. In particular, it can be provided that the second imaging data includes second vascular data and/or second parenchyma data, wherein the second vascular data relates to the vascular structure at the second point in time, wherein the second parenchyma data relates to the parenchyma at the second point in time.

One embodiment provides that the first imaging data is based on a first contrast medium imaging of the vascular structure and/or on a first contrast medium imaging of the parenchyma and/or that the second imaging data is based on a second contrast medium imaging of the vascular structure and/or on a second contrast medium imaging of the parenchyma. The first contrast medium imaging and/or the second contrast medium imaging can in particular be based on a contrast medium which flows through the vascular structure and/or the parenchyma together with the blood.

One embodiment provides that the supply to the parenchyma is a blood supply to the parenchyma and/or an oxygen supply to the parenchyma.

One embodiment provides that, in particular for the third point in time, a simulation of the flow dynamics of the vascular structure and/or the perfusion dynamics of the parenchyma is calculated based on the first imaging data and the reference data, in particular the reference data in the form of the second imaging data, wherein the supply data are calculated based on the simulation. The simulation can be calculated in particular for third blood pressure data, which is assumed for the third point in time.

An embodiment of the present invention further relates to a data processing system for providing supply data relating to a supply of a parenchyma, comprising a data interface and a processor, wherein the data processing system is set up to carry out a method according to embodiments of the present invention.

An embodiment of the present invention further relates to a medical imaging system, comprising the data processing system according to an embodiment of the present invention and a medical imaging device for recording the first imaging data. The medical imaging device can, for example, be a computer tomography device and/or be set up to record the second imaging data.

An embodiment of the present invention further relates to a computer program product comprising instructions which, when the instructions are executed by a computer, cause the computer to carry out the method according to an embodiment of the present invention. The method according to embodiments of the present invention can in particular be computer-implemented.

The computer program product can, for example, be a computer program or include at least one additional component in addition to the computer program. The at least one additional component of the computer program product can be designed as hardware and/or as software.

The computer program product can, for example, have a storage medium on which at least part of the computer program product is stored and/or a key for authenticating a user of the computer program product, in particular in the form of a dongle. The computer program product and/or the computer program can, for example, have a cloud application program which is designed to distribute the instructions to different processing units, in particular different computers, of a cloud computing system, wherein each of the processing units is designed to execute one or more of the instructions.

An embodiment of the present invention further relates to a computer-readable storage medium comprising instructions which, when the instructions are executed by a computer, cause the computer to carry out the method according to an embodiment of the present invention.

The method can combine morphological, functional and simulated information. The morphological information can be determined from the CTA, for example. The functional information includes both non-image-supported information (blood pressure) and image-supported information (vessel diameter, blood pool) and can be supplemented with a priori generated simulations on certain aspects of the vascular supply (flow behavior, flow capacity). Furthermore, standard variants, which can be represented in particular in a model, and/or the cerebral autoregulation of blood flow in the brain wave zone can be taken into account.

Depending on the vascular situation (e.g. calcification), different mechanisms (change in blood pressure, change in vessel diameter) are used to generate different measurement states from which the effects of changes in blood flow can be calculated. In particular, this result can also be achieved with a one-time imaging examination and the use of reference data in the form of a priori knowledge from its database.

In particular, the blood flow in the vascular structure can be recorded. This is done, for example, by measuring blood pressure (upper arm on both sides as a surrogate parameter) immediately before a CTA. The contrast medium imaging can take place in particular in the form of a CTA of the aortic arch up to the cerebral vessels, whereby the contrast medium amount, concentration, flow and the injection location are known from the imaging. Furthermore, a segmentation (diameter) of the left and right neck flow zone and/or a measurement of the signal intensity at predefined points in both the inflow and the basal cerebral vessels can be carried out. The flow dynamics can be simulated in particular via the detected and segmented vascular tree, especially including the Circle of Willis.

A delayed (8-10 s) contrast medium imaging of the brain parenchyma can be performed to assess the distribution of the blood pool in the terminal flow zone/brain parenchyma, particularly following CTA, particularly in the form of a gradient measurement. A change in the cerebral perfusion dynamics can occur, for example, by changing the diameter of the cerebral vessels with medication and/or by increasing or decreasing the arterial systemic blood pressure, in particular so that when the second imaging data is recorded, another flow dynamic of the vascular structure and/or another perfusion dynamic of the parenchyma is present than when the first imaging data was recorded. In particular, further sequential repetitions are possible with different blood pressures and/or vessel diameters.

Furthermore, differences between the first imaging data and the second imaging data with respect to the flow dynamics of the vascular structure and/or the perfusion dynamics of the parenchyma can be determined. Based on this, a simulation of the effect of a change in blood pressure on perfusion can be calculated, in particular based on the values determined for the first point in time and the second point in time. For follow-up checks or if correspondingly large databases are available, it is sufficient to carry out the first contrast medium imaging in a delayed CTA in conjunction with a subsequent comparison of the first imaging data with the reference data from a database. Particularly for follow-up checks after an intervention, repeating the measurement with and/or without a change in blood pressure can be useful. The method can also use a priori knowledge based on databases. This would also make it possible to correct vascular simulations as part of a digital patient twin.

Particularly in the case of existing autoregulation (lack of calcification of the vessels), the signal intensity can be measured, particularly in the form of the SI behavior, at one or more defined end points of the visible vascular tree behind the stenosis. By knowing the amount of contrast medium injected, the vessel diameter and the density and iodine concentration present at the measuring point, it can be determined to what extent the maximum vessel flow volume has been used. The comparison with a priori knowledge (database) provides a statement as to whether the blood flow at this point is sufficient to adequately supply the parenchyma behind it.

If this is not the case and there are no signs of ischemia in the relevant area, it must be assumed that there is sufficient collateralization via the adjacent vessel. This method could also be used to exclude relevant anomalies of the vascular system. If there is a potentially hemodynamically relevant stenosis, suspected impaired regulation and/or extensive collateralization, a delayed contrast medium uptake would be carried out. This takes place in such a way that it corresponds to the arterialized blood pool of the parenchyma.

The gradients can be measured across the vascular territories as a measure of the saturation of the parenchyma. Inhomogeneity indicates reduced perfusion and insufficient collateralization in a vessel.

If insufficient autoregulation is suspected (e.g. existing vascular calcifications, also automatically detectable through calcium segmentation using photon counter CT), this model can no longer be easily applied. In this context, the actual reserve of the end flow zone is highly dependent on blood pressure and the resulting blood flow. In particular, in such a case, the second imaging data can be recorded after an artificial reduction in blood pressure and/or a change in the vessel diameter. In a further step, it would also be conceivable to determine the transport capacity for oxygen in the blood and thus also the oxygen saturation present in the parenchyma or the reserve capacity there by determining the hemoglobin content and the oxygen saturation.

Based on the first imaging data and the reference data, a simulation can be calculated for the patient, which describes, for example, the blood flow behavior at different, in particular not previously measured, values of the vascular parameters. This involves either interpolation or extrapolation or access to existing, comparable patient data in a database. The newly acquired patient data can then also be entered into the database and/or used for a digital twin.

This method provides an option for cases in which a conventional FFR calculation would provide inadequate results. Although the main application in this specific case is the brain, this method could also be used to evaluate the influence of collateralization on an end flow zone. Further examples would be liver blood flow after TIPS placement or evaluation of cardiac bypass.

The method can in particular include a determination of the flow conditions in front of and behind the stenosis. From this it can be estimated from which vessels (main vessels or collaterals) and to what extent the main supply to the adjoining zone comes. Furthermore, on the basis of the supply data, an assessment of the effect of changes in the blood circulation situation on the downstream flow zone and/or an assessment of the risk of a future under-supply of the parenchyma can be made.

For example, the computer program product according to one of the embodiments disclosed in this application and/or the computer program according to one of the embodiments disclosed in this application can be stored on the computer-readable storage medium. The computer-readable storage medium can be, for example, a memory stick, a hard drive or another data carrier, which can in particular be detachably connected to a computer or permanently integrated into a computer. The computer-readable storage medium can, for example, form a sector of a storage system, wherein the data processing system is connected to the storage system via the data interface.

The data processing system can, for example, have one or more components in the form of hardware and/or one or more components in the form of software. The data processing system can, for example, be at least partially formed by a cloud computing system. The data processing system can be and/or have, for example, a cloud computing system, a computer network, a computer, a tablet computer, a smartphone or the like or a combination thereof.

The hardware can, for example, interact with software and/or be configurable using software. The software can be executed using the hardware, for example. The hardware can be, for example, a memory system, an FPGA (field-programmable gate array) system, an ASIC (application-specific integrated circuit) system, a microcontroller system, a processor system and combinations thereof. The processor system can, for example, have a microprocessor and/or a plurality of interacting microprocessors.

The steps of the method can be carried out, for example, in the processor of the data processing system, in particular in the form of calculations. A calculation, for example calculating the supply data, can be carried out in particular by applying an algorithm, for example a trained machine learning algorithm, to the data on which the calculation is based.

A data transfer between components of the medical imaging system can, for example, take place using a suitable data transfer interface. The data transfer interface for data transfer to and/or from a component of the medical imaging system can be realized at least partially in the form of software and/or at least partially in the form of hardware. The data transfer interface can, for example, be designed to store data in and/or read data from a sector of a storage system, wherein one or more components of the medical imaging system are able to access this sector of the storage system.

Data, in particular the first imaging data, the second imaging data and the reference data, can be received, for example, by receiving a signal that carries the data and/or by reading the data, in particular reading it from a computer-readable storage medium. Data, in particular the supply data, can be provided, for example, by transmitting a signal carrying the data and/or by writing the data into a computer-readable storage medium and/or by displaying the data on a screen.

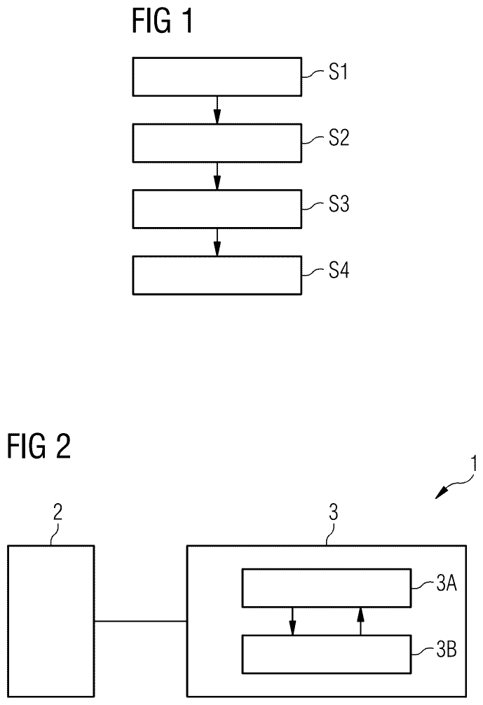

FIG. 1 shows a flowchart of a method for providing supply data relating to the supply of a parenchyma, the method comprising:
receiving S1 first imaging data, wherein the first imaging data relates to a vascular structure that serves to supply the parenchyma, and/or the parenchyma,
receiving S2 reference data for parenchyma supply,
calculating S3 the supply data, wherein the supply data relates to the supply of the parenchyma, based on the first imaging data and the reference data,
providing S4 the supply data.

FIG. 2 shows a medical imaging system 1, comprising the data processing system 3 and a computer tomography device 2 for recording the spectral computer tomography data. The data processing system 3 has a data interface 3A and a processor 3B and is set up to carry out the method shown in FIG. 1.

Within the scope of the present invention, features that are described in relation to different embodiments of the present invention and/or different claim categories (method, use, device, system, arrangement, etc.) can be combined to form further embodiments of the present invention. For example, a claim that relates to a device can also be developed with features that are described or claimed in connection with a method and vice versa. Functional features of a method can be executed by appropriately designed physical components. The use of the indefinite article “a” or “an” does not exclude the possibility that the feature in question can exist multiple times. In the context of the present application, the expression “based on” can be understood in particular within the meaning of the expression “using”. In particular, a formulation according to which a first feature is calculated (alternatively: determined, generated, etc.) based on a second feature does not exclude that the first feature can be further calculated (alternatively: determined, generated, etc.) based on a third feature.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element (s) or feature (s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A method for providing supply data relating to a supply to a parenchyma, the method comprising:
receiving first imaging data, wherein the first imaging data relates to at least one of the parenchyma or a vascular structure that serves to supply the parenchyma;
receiving reference data for the supply to the parenchyma;
calculating supply data based on the first imaging data and the reference data, wherein the supply data relates to the supply to the parenchyma; and
providing the supply data.

2. The method according to claim 1, wherein the vascular structure is ring-shaped.

3. The method according to claim 1,
wherein the reference data includes second imaging data,
wherein, at a first point in time, the first imaging data relates to at least one of the vascular structure or the parenchyma, and
wherein, at a second point in time, the second imaging data relates to at least one of the vascular structure or the parenchyma.

4. The method according to claim 3, wherein at least one of
flow dynamics of the vascular structure at the first point in time differ from the flow dynamics of the vascular structure at the second point in time, or
perfusion dynamics of the parenchyma at the first point in time differ from the perfusion dynamics of the parenchyma at the second point in time.

5. The method according to claim 3, wherein the vascular structure at the first point in time differs from the vascular structure at the second point in time with respect to at least one of a blood pressure or a vessel diameter.

6. The method according to claim 5, further comprising:
receiving first blood pressure data and second blood pressure data, the first blood pressure data relating to the blood pressure at the first point in time, and the second blood pressure data relating to the blood pressure at the second point in time, and
comparing the first blood pressure data with the second blood pressure data.

7. The method according to claim 5, further comprising:
calculating a first vessel diameter of the vascular structure at the first point in time, based on the first imaging data,
calculating a second vessel diameter of the vascular structure at the second point in time, based on the second imaging data, and
comparing the first vessel diameter with the second vessel diameter.

8. The method according to claim 3,
wherein the supply data relates to the supply to the parenchyma at a third point in time, and
wherein the third point in time is after the first point in time and the second point in time.

9. The method according to claim 3, wherein at least one of
the first imaging data is based on a first contrast medium imaging of the vascular structure and on a first contrast medium imaging of the parenchyma, or
the second imaging data is based on a second contrast medium imaging of the vascular structure and on a second contrast medium imaging of the parenchyma.

10. The method according to claim 1, wherein the supply to the parenchyma includes at least one of a blood supply to the parenchyma or an oxygen supply to the parenchyma.

11. The method according to claim 1, further comprising:
calculating, based on the first imaging data and the reference data, a simulation of at least one of flow dynamics of the vascular structure or perfusion dynamics of the parenchyma, and wherein
the supply data is calculated based on the simulation.

12. A data processing system for providing supply data relating to a supply to a parenchyma, the data processing system comprising:
a data interface and a processor, wherein the data processing system is configured to perform the method according to claim 1.

13. A medical imaging system comprising:
the data processing system according to claim 12; and
a medical imaging device configured to record the first imaging data.

14. A non-transitory computer program product including instructions which, when executed by a computer, cause the computer to carry out the method according to claim 1.

15. A non-transitory computer-readable storage medium storing instructions which, when executed by a computer, cause the computer to carry out the method according to claim 1.

16. The method according to claim 2,
wherein the reference data includes second imaging data,
wherein, at a first point in time, the first imaging data relates to at least one of the vascular structure or the parenchyma, and
wherein, at a second point in time, the second imaging data relates to at least one of the vascular structure or the parenchyma.

17. The method according to claim 4, wherein the vascular structure at the first point in time differs from the vascular structure at the second point in time with respect to at least one of a blood pressure or a vessel diameter.

18. The method according to claim 6,
calculating a first vessel diameter of the vascular structure at the first point in time, based on the first imaging data,
calculating a second vessel diameter of the vascular structure at the second point in time, based on the second imaging data, and
comparing the first vessel diameter with the second vessel diameter.

19. The method according to claim 7,
wherein the supply data relates to the supply of the parenchyma at a third point in time, and
wherein the third point in time is after the first point in time and the second point in time.

20. The method according to claim 8, wherein at least one of
the first imaging data is based on a first contrast medium imaging of the vascular structure and on a first contrast medium imaging of the parenchyma, or
the second imaging data is based on a second contrast medium imaging of the vascular structure and on a second contrast medium imaging of the parenchyma.
